(19) United States
(12) Patent Application Publication
(10) Pub. No.: US 2026/0198890 A1
(43) Pub. Date: Jul. 16, 2026

(54) SYSTEMS AND METHODS FOR DETECTING SOUND IN A BONE

(71) Applicant: DePuy Synthes Products, Inc., Raynham, MA (US)

(72) Inventor: Jeffrey Bizub, Greenfield, WI (US)

(73) Assignee: DePuy Synthes Products, Inc., Raynham, MA (US)

(21) Appl. No.: 19/444,636

(22) Filed: Jan. 9, 2026

Related U.S. Application Data
(60) Provisional application No. 63/743,795, filed on Jan. 10, 2025.

(51) Int. Cl. A61B 8/08; A61B 8/12

(52) U.S. Cl. CPC A61B 8/0875; A61B 8/12

(57) ABSTRACT

A system may include an emitter. The emitter may be configured to generate and/or apply sound waves, for example to a first end of a patient's bone. The emitter may be one or more of a surface transducer, a sound emitter, a speaker, and/or a piezoelectric device. The system may additionally, or alternatively, include a receiver. The receiver may be configured to detect sound waves, for example at a second end of the bone. For example, the receiver may detect sound waves (e.g., at the second end of the bone) after the sound waves propagate through the bone. The receiver may be one or more of a microphone, a piezoelectric device, and/or an accelerometer.

### CROSS-REFERENCE TO RELATED APPLICATIONS

This application claims the benefit of U.S. Provisional Ser. No. 63/743,795, filed on Jan. 10, 2025, the contents of which are incorporated by reference herein in its entirety.

### BACKGROUND

In a medical context, an indication of bone health may be useful. The indication of bone health may be useful for a surgical procedure, for example spinal surgery. Information may be required to avoid harming the patient by damaging a bone.

Existing solutions for determining bone health may not be available prior to, during, and after a surgical procedure. Additionally, or alternatively, existing solutions may not provide sufficient information to avoid bone damage. Within this context, there is a need for determining bone health.

### SUMMARY

The application is generally related to devices and methods for detecting sound and/or vibration, for example in a medical context, and more particularly, to detecting sound and/or vibration in a bone.

A system may include an emitter. The emitter may be configured to generate and/or apply sound waves, for example to a first end of a patient's bone. The emitter may be one or more of a surface transducer, a sound emitter, a speaker, and/or a piezoelectric device. The system may additionally, or alternatively, include a receiver. The receiver may be configured to detect sound waves, for example at a second end of the bone. For example, the receiver may detect sound waves (e.g., at the second end of the bone) after the sound waves propagate through the bone. The receiver may be one or more of a microphone, a piezoelectric device, and/or an accelerometer. The receiver may measure, record, and/or analyze sound waves (e.g., signals) with a frequency from 0 -150 kHz in some examples. For example, the receiver may measure, record, and/or analyze sound waves (e.g., signals) in an ultrasonic range and/or a human hearing range. A sideband in the sound wave (e.g., signal) may indicate a location and/or a bone quality, for example a degradation of bone quality (e.g., at a location and/or time).

The system may include an attachment mechanism. The attachment mechanism may be configured to connect the receiver and/or the emitter to the bone. For example, the attachment mechanism may be positioned to a specific area of a bone. The attachment mechanism may include one or more of a clamp, an adhesive, and/or a screw.

The system may include a circuit. The circuit may be configured to collect data from one or more of the emitter and/or the receiver. The circuit may compare the sound waves applied at the first end of the bone to the detected sound waves at the second end of the bone, for example to determine an indication of bone quality. The circuit may be configured to determine the indication of bone quality, for example based on an acoustic impedance of the sound waves propagated through the bone. The acoustic impedance of the sound waves may include a function of one or more of a force, stress, and/or load, for example applied to the bone. The circuit may determine a location and/or bone quality, for example by measuring, recording, and/or analyzing a sound wave. For example, the circuit may measure, record, and/or analyze a sideband in a sound wave (e.g., signal). The sideband in the sound wave (e.g., signal) may indicate a location and/or a bone quality, for example a degradation of bone quality (e.g., at a location and/or time).

The circuit may be configured to determine the indication of bone quality based on at least one of the sound waves applied at the first end of the bone or the detected sound waves at the second end of the bone. The indication of bone quality may include one or more of splintering, fracturing, cracking, and/or breaking. Additionally, or alternatively, the system may include a filtering circuit. The filtering circuit may be a 4.9 to 5.1 kHz bandpass filter. The system may include a display. The display may be configured to display an indication of one or more of the sound waves applied at the first end of the bone and/or the detected sound waves at the second end of the bone. The indication of the one or more sound waves may include a graph.

Additional features and advantages are realized through the system of the present invention. Other embodiments and aspects of the disclosure are described in detail herein. For a better understanding of the disclosure with advantages and features, refer to the description and to the drawings.

### BRIEF DESCRIPTION OF THE DRAWINGS

Many aspects of the disclosure can be better understood with reference to the following drawings. The components in the drawings are not necessarily to scale, with emphasis being placed upon clearly illustrating the principles of the present disclosure. Moreover, in the drawings, like reference numerals designate corresponding parts throughout the several views. Furthermore, each drawing contained in this application includes at least a brief description thereon and associated text labels further describing associated details.

FIG. 1 is a view of an example of a intervertebral implant.

FIG. 2 is a view of an intervertebral implant in a collapsed position.

FIG. 3A is a view of an example of an intervertebral implant positioned between adjacent vertebral bodies.

FIG. 3B is a view of the intervertebral implant positioned between adjacent vertebrae as in FIG. 3A.

FIG. 4 is a sectional view of an example system for applying and detecting sound.

FIG. 5 is another sectional view of an example system for applying and detecting sound in a bone.

FIG. 6 is an example graph of data indicating bone quality.

FIG. 7 is an example graph of data generated using a bandpass filter.

FIG. 8 includes example graphs of data indicating spine failure.

FIG. 9 includes additional example graphs of data indicating spine failure.

### DETAILED DESCRIPTION

Herein, example embodiments of the present disclosure will be described in detail. Example embodiments of the present disclosure provide a system and method for detecting sound as described below.

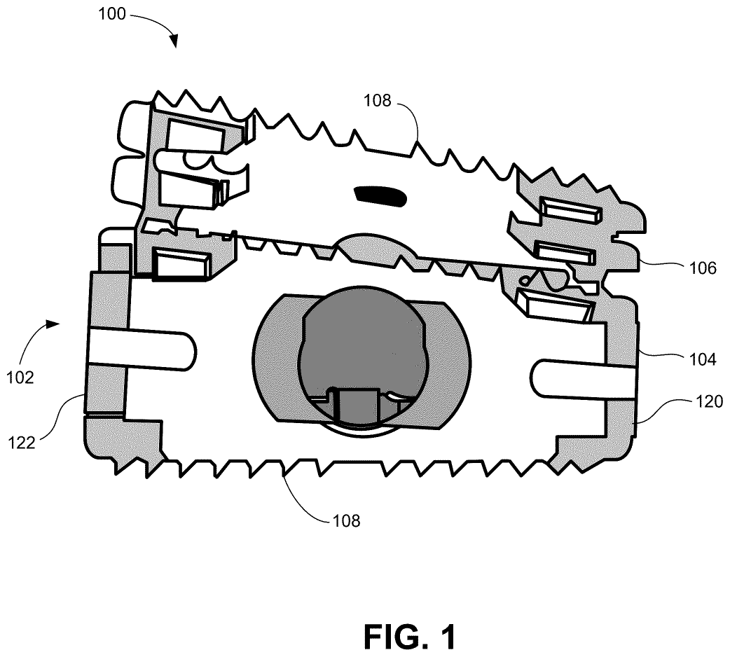

FIG. 1 is a view of an example of an intervertebral implant 100. The intervertebral implant may be inserted between a patient's vertebrae, for example to establish a desired spacing between adjacent vertebrae. The intervertebral implant includes an expandable cage 102. The expandable cage may include a base portion 104 and/or an expansion portion 106. A lifting mechanism may be included in the expandable cage 102 to actuate the expansion portion 106. The lifting mechanism may include an actuator, for example as described in US Patent Publication No. 2023/0277329A1 entitled “Stabilization members for expandable intervertebral implants, and related systems and methods”, which is hereby incorporated by reference. The base portion 104 may include locking teeth 108, for example unidirectional locking teeth. Additionally, or alternatively, the expansion portion 106 may include locking teeth 108. The locking teeth may be configured to grip a bone, for example a vertebrae.

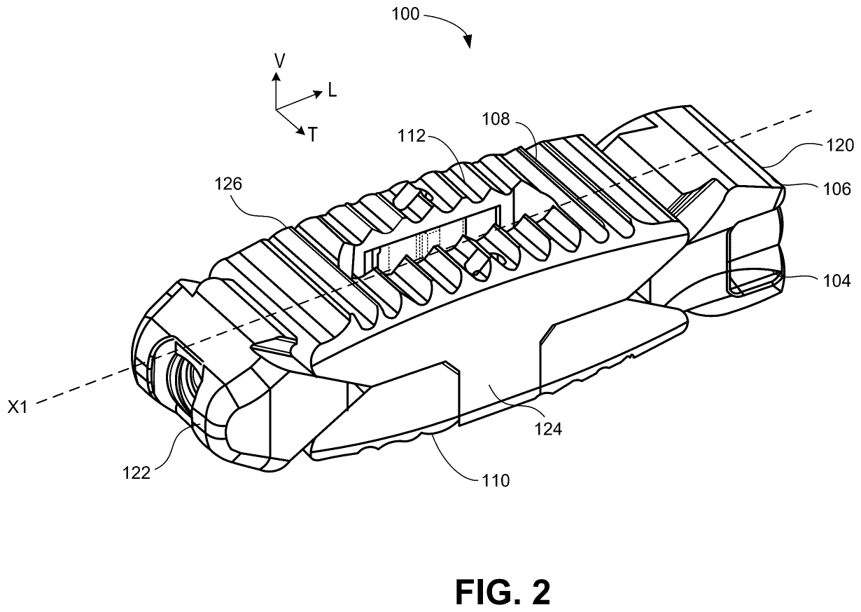

FIG. 2 is a view of the intervertebral implant 100 of FIG. 1 in a collapsed position. The intervertebral implant 100 may extend between a first end 120 and a second end 122. The first end 120 may be spaced from the second end 122 along a longitudinal implant axis X1 that extends along the longitudinal direction L. The intervertebral implant 100 may be inserted by first inserting the first end 120 into an intervertebral space. The second end 122 may be configured to couple with one or more insertion tools. The one or more insertion tools may be configured to support and/or carry the intervertebral implant 100 into the intervertebral space. The intervertebral implant 100 may additionally, or alternatively, extend between a first side 124 and an opposed second side 126, for example along the transverse direction T.

The intervertebral implant 100 may include a base portion plate 110 and an extension portion plate 112 opposing the base portion plate 110, for example along the vertical direction V. The extension portion plate 112 and/or the base portion plate 110 may be configured to contact a bone, for example with locking teeth. The extension portion plate 112 and/or the base portion plate 110 may be configured to engage the opposing adjacent vertebrae. The extension portion plate 112 and/or the base portion plate 110 may extend in a substantially convex fashion. For example, the extension portion plate 112 and/or the base portion plate 110 may be convex along both the longitudinal and transverse directions L, T. In other examples, the extension portion plate 112 and/or the base portion plate 110 may have a convex profile along only one of the longitudinal and transverse direction L, T. In some examples, the extension portion plate 112 and/or the base portion plate 110 may be substantially planer.

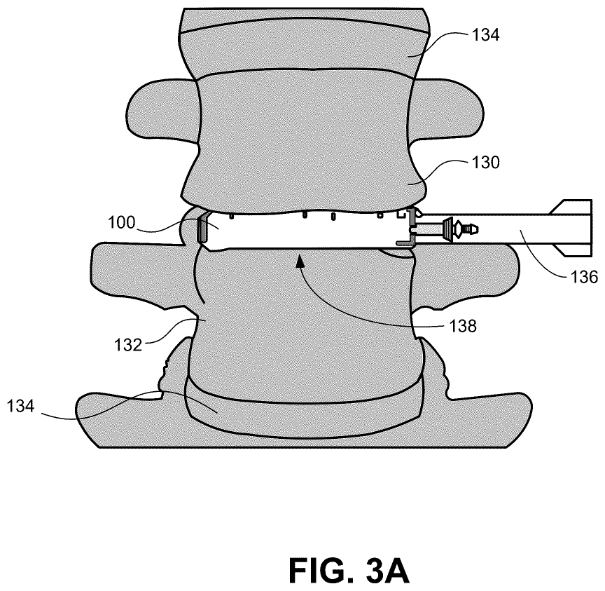

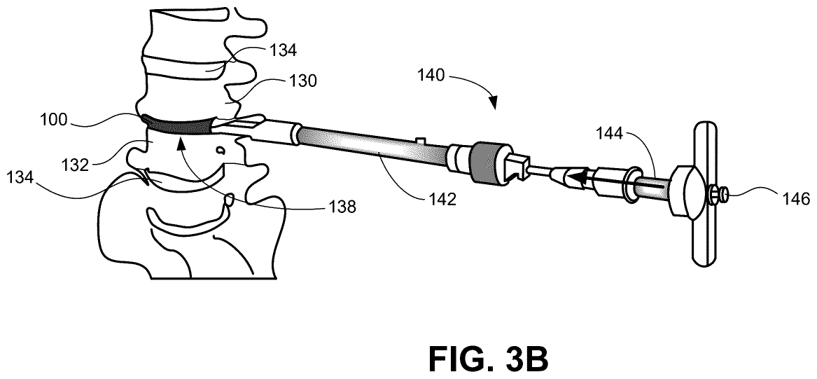

FIG. 3A is a view of an example of the intervertebral implant 100 of FIG. 1 positioned between adjacent vertebral bodies. The intervertebral implant 100 may be inserted between a first vertebrae 130 and a second vertebrae 132. The intervertebral implant 100 may be positioned in place of a disc 134. The intervertebral implant 100 may be positioned in place of the disc 134 using one or more insertion tools. An example insertion tool 136 may be configured to support and/or carry the intervertebral implant 100 into an intervertebral space 138 (e.g., in place of a disc 134).

Additionally, or alternatively, an insertion tool may be configured to actuate the expansion portion of the intervertebral implant 100. For example, the insertion tool may be rotatable such that rotation in one direction (e.g., clockwise) actuates the expansion portion to expand and/or rotation in another direction (e.g., counter-clockwise) actuates the expansion portion to contract (e.g., toward the base portion). The insertion tool 136 may include a knob. The knob may be actuated in one direction (e.g., clockwise) to actuate the expansion portion to expand and/or in another direction (e.g., counter-clockwise) to actuate the expansion portion to contract.

A user may use the insertion tool to increase a height of the intervertebral implant 100 until a desired height is reached within the intervertebral space 138. Once the desired height has been reached for example, the user may rotate the know (e.g., counter-clockwise) one complete rotation to release tension in the insertion tool 136. Additionally, or alternatively, the intervertebral implant 100 may include a locking mechanism configured to lock the intervertebral implant 100 to the insertion tool 136. For example, a user may switch a toggle switch on the insertion tool 136 to a locked and an unlocked position. The user may remove the insertion tool 136 from the intervertebral implant 100 when the toggle switch is in the unlocked position.

FIG. 3B is a view of the intervertebral implant positioned between adjacent vertebrae, for example as in FIG. 3A. The intervertebral implant 100 may be positioned between the first vertebrae 130 and the second vertebrae 132, for example in the intervertebral space 138 (e.g., in place of a disc 134). A removal tool 140 may be configured to remove the intervertebral implant 100 from the intervertebral space 138. The removal tool may include a shaft 142 and/or a handle 144. The shaft 142 may be inserted into the intervertebral implant 100. The user may rotate the handle 144 to lock in place with respect to the intervertebral implant 100.

The handle may be configured to connect to a slap hammer. The user may slide the slap hammer onto a cap 146 of the handle 144. The user may contract the intervertebral implant 100 by sliding the slap hammer toward the cap 146 of the handle 144. Once the intervertebral implant 100 is in a collapsed position for example, the user may remove the intervertebral implant 100 from the intervertebral space 138 of the patient.

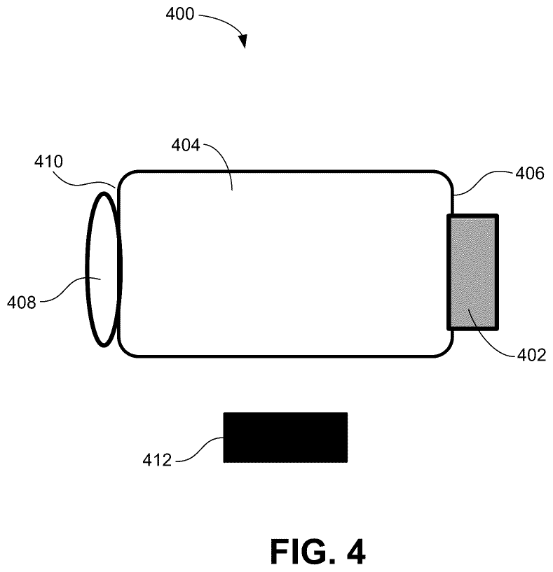

FIG. 4 is a sectional view of an example system 400 for applying and detecting sound waves. Although the systems and methods herein are described utilizing sound waves, the systems and methods may additionally, or alternatively, be utilized to apply and/or detect vibrations, for example similarly as described for sounds waves. The system 400 may include an emitter 402. The emitter 402 may be configured to generate and/or apply sound waves, for example at a (e.g., center) frequency. For example, the emitter 402 may apply sound waves at a patient's bone 404. The emitter 402 may apply a sound wave at a first end 406 of the bone. The emitter 402 may include one or more of a surface transducer, a sound emitter, a speaker, and/or a piezoelectric device.

The system 400 may be used with an intervertebral implant, such as the intervertebral implant 100 of FIG. 1. For example, the user may place the intervertebral implant between vertebrae. Additionally, or alternatively, the user may apply the system 400 at one or more vertebrae of a patient. The user may actuate the intervertebral implant (e.g., the expansion portion may be actuated) to produce one or more of a force, stress, and/or load to the bone (e.g., vertebrae).

The system 400 may include a receiver 408 that may be configured to detect sound waves, for example at a second end 410 of the bone 404. The receiver 408 may detect a sound wave (e.g., at the second end 410 of the bone 404), for example after the sound waves propagate through the bone. The receiver 408 may include one or more of a microphone, a piezoelectric device, and/or an accelerometer. The receiver may measure, record, and/or analyze sound waves (e.g., signals) with a frequency from 0 -150 kHz in some examples. For example, the receiver may measure, record, and/or analyze sound waves (e.g., signals) in an ultrasonic range and/or a human hearing range. A sideband in the sound wave (e.g., signal) may indicate a location and/or a bone quality, for example a degradation of bone quality (e.g., at a location and/or time).

The system 400 may include an attachment mechanism (not shown). The attachment mechanism may be configured to connect the emitter 402 to the bone 404, for example at the first end 406. Additionally, or alternatively, the attachment mechanism may be configured to connect the receiver 408 to the bone 404, for example at the second end 410. Example attachment mechanisms may include one or more of a clamp, an adhesive, and/or a screw.

The system 400 may include a circuit 412 that includes a processor and memory. The processor may include one or more of a microprocessor, a microcontroller, a programmable logic device (PLD), a field programmable gate array (FPGA), an application specific integrated circuit (ASIC), and/or any suitable controller or processing device. The memory may be communicatively coupled to the processor for the storage and/or retrieval of, for example, operational settings of the system 400. The memory may be implemented as an external integrated circuit (IC) or as an internal circuit of the circuit 412. The memory may comprise a computer-readable storage media or machine-readable storage media that maintains computer-executable instructions for performing one or more procedure and/or functions as described herein. Additionally, or alternatively, the circuit 412 may include an analog circuit.

The circuit 412 may be configured to collect data from one or more of the emitter 402 and/or the receiver 408. For example, the circuit 412 may collect input sound data from a sound wave generated by the emitter 402 (e.g., at the first end 406 of the bone 404). Additionally, or alternatively, the circuit 412 may collect output sound data from a sound wave detected by the receiver 408 (e.g., at the second end 410 of the bone 404). The receiver 408, the emitter 402, and/or the circuit 412 may be in communication with one another. The receiver 408, the emitter 402, and/or the circuit 412 may be configured to receive and/or transmit a signal, for example with a wired or wireless communication link. Examples of communication links may include one or more of a radio frequency (RF) communication link, a Wi-Fi communication link, a Wi-MAX communications link, a Bluetooth communications link, a near field communication (NFC) link, a cellular communications link, and/or a television white space (TVWS) communication link. Additionally, or alternatively, the receiver 408, the emitter 402, and/or the circuit 412 may be configured to receive and/or transmit a signal via a wired connection.

The circuit may determine a location and/or bone quality, for example by measuring, recording, and/or analyzing (e.g., processing) a sound wave. For example, the circuit may measure, record, and/or analyze a sideband in a sound wave (e.g., signal). The sideband in the sound wave (e.g., signal) may indicate a location and/or a bone quality, for example a degradation of bone quality (e.g., at a location and/or time).

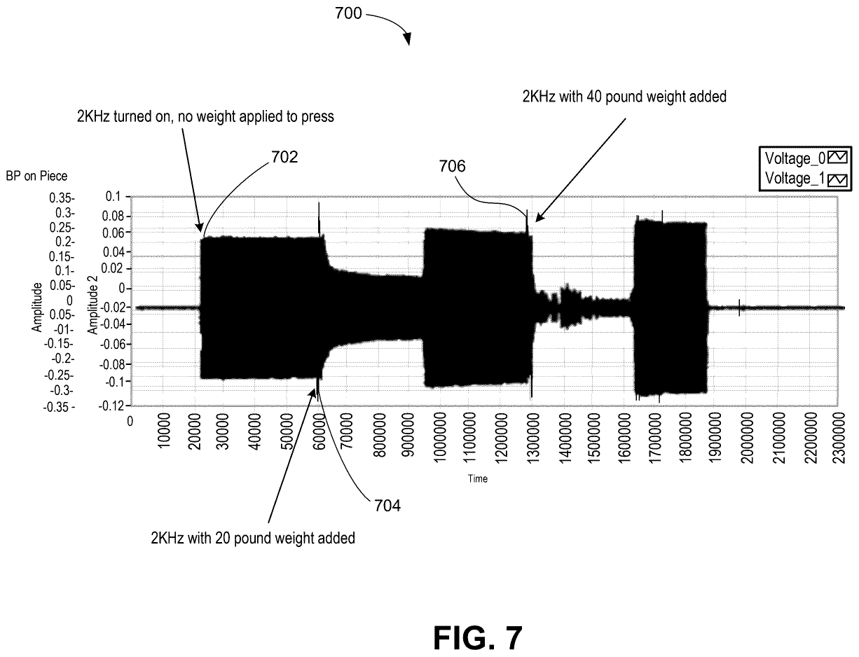

The circuit 412 may process one or more of the input sound data and/or the output sound data, for example sideband data. For example, the circuit 412 may measure, record, and/or analyze sound waves (e.g., signals) with a frequency from 0 -150 kHz. The circuit may compare the input sound data and the output sound data, for example to determine an indication of bone quality. The circuit 412 may be configured to determine the indication of bone quality based on at least one of the sound waves applied at the first end of the bone or the detected sound waves at the second end of the bone, for example, as described in more detail below with respect to FIGS. 7, 8, and 9. The indication of bone quality may include one or more of splintering, fracturing, cracking, and/or breaking. For example, the circuit may process a sound wave over an amount of time and/or one or more positions (e.g., location) of the sound wave.

The sound wave and/or vibration may be applied to a bone of a patient. The circuit 412 may perform measurements of the sound waves during a (e.g., surgical) procedure, before a (e.g., surgical) procedure, and/or after a (e.g., surgical) procedure. The circuit 412 may determine an indication of bone quality based on the measured sound waves, for example, because a degradation of bone quality (e.g., at a location and/or time) may affect the sound waves. The circuit 412 may provide the measurement (e.g., of bone quality), for example to the user (e.g., physician, during the (e.g., surgical) procedure, before the (e.g., surgical) procedure, and/or after the (e.g., surgical) procedure, for instance, via a user interface such as a display device (not shown). The user may make adjustments to the procedure, for example based on the measurement. The user may make the adjustments by changing one or more of the emitter 402, the receiver 408, the circuit 412. For example, the user may adjust settings or programming of the circuit. The user may make adjustments using the user interface. Additionally, or alternatively, the user may adjust the relative position of one or more of the emitter 402, the receiver 408, the circuit 412, and/or the bone 404.

The circuit 412 may be configured to determine the indication of bone quality, for example based on one or more sound metrics. For example, the circuit 412 may determine a sound metric. The sound metric may include one or more of an amplitude, a frequency, and/or an impedance of the sound wave. A sound metric may be based on the sound data, for example, based on input sound data and/or output sound data. Input sound data may be based on sound waves generated by the emitter 402. Output sound data may be based on sound waves received by the receiver 408.

The sound metric may include the amplitude, frequency, and/or impedance of the detected sound wave. Additionally, or alternatively, the circuit 412 may determine the sound metric to be a difference between the emitted sound wave (e.g., at the second end of the bone) and the applied sound wave (e.g., at the first end of the bone). For example, the sound metric may include a difference in the amplitude, frequency, deflection, and/or impedance between the emitted sound wave and the applied sound wave. Deflection (e.g., sound deflection) may change as a bone compresses or decompresses. For example, as a bone compresses more sound (e.g., energy) may be reflected which may result in less (e.g., sound) propagation through the bone (e.g., impedance).

The sound metric may additionally, or alternatively, be based on (e.g., a function of) one or more of a force, stress, compression, and/or load, for example applied to the bone. For example, the circuit 412 may determine a sound metric based at least partially on one or more of a force, stress, and/or load applied to the bone. The circuit 412 may determine the compression (e.g., value), for example based on deflection. For example, the circuit 412 may determine the compression (e.g., value) based on the amplitude, frequency, and/or impedance of the detected sound wave (e.g., associated with the deflection). The circuit 412 may determine the force applied to the bone based on the compression value. The force, stress, and/or load may be predetermined (e.g., selected by a physician). Additionally, or alternatively, the sound metric may be based on a time and/or position (e.g., location) of the sound wave. For example, a sound impedance value (e.g., at a location in the bone) may indicate a degradation of bone quality. The sound metric may be based on one or more of the input sound data, output sound data, force, stress, and/or load.

The sound output data and/or the sound metric may be received directly from the receiver 408 (e.g., microphone). For example, the receiver 408 (e.g., microphone) may receive a sound wave (e.g., a sound wave metric) that indicates a bone quality change and/or degradation. The circuit 412 may measure the sound waves. The circuit may make measurements on the input sound data, the output sound data, and/or the sound metric. Alternatively, or additionally, the circuit 412 may process the sound input data, the sound output data and/or sound metric. For example, the circuit 412 may filter and/or process a sound wave (e.g., output sound data) signal. The circuit may process the sound input data, the sound output data and/or sound metric to determine the bone quality change and/or degradation, for example based on a difference between the sound input data and the sound output data.

The circuit 412 may determine a discontinuity, distortion, crack formation (e.g., initial), and/or point of failure based on one or more of an amplitude, a frequency, and/or impedance. The circuit 412 may determine a discontinuity, distortion, crack formation (e.g., initial), and/or point of failure based on a difference of amplitude, frequency, and/or impedance, for example between the emitted sound wave and the applied sound wave. For example, the circuit 412 may determine the discontinuity, distortion, crack formation (e.g., initial), and/or point of failure when the difference of amplitude, frequency, and/or impedance exceeds a threshold value.

The circuit 412 may determine the discontinuity, distortion, crack formation, and/or point of failure by determining (e.g., detecting) phantom beats and/or notes. For example, the circuit 412 may determine terzo suono in the bone. The circuit 412 may determine stress (e.g., on the bone) based on an amount of discontinuity, distortion, crack formation, and/or point of failure. For example, the stress may include one or more of compression, yielding, stretching, and/or fracturing.

Processing may include signal conditioning and/or digital signal processing, for example to determine a sound metric. The circuit 412 may process the sound input data, the sound output data and/or sound metric by time and/or position (e.g., location in a bone). Additionally, or alternatively, the circuit 412 may include a filtering circuit. The filtering circuit may be an analog and/or digital filtering circuit. For example, the filtering circuit may be a 4.9 to 5.1 kHz bandpass filter. The circuit 412 may compare the sound input data to the sound output data, for example at one or more frequencies. Frequencies may be in the range of 0.1 Hz to 25,000 Hz. For example, the frequencies may be in the range of 3 kHz to 500 Hz. Additionally, or alternatively, the circuit 412 may process sound input data, sound output data and/or sound metric both with the filtering circuit and without the filtering circuit. The circuit 412 may analyze (e.g., compare) the sound input data, the sound output data and/or sound metric with the filtering circuit and without the filtering circuit, for example to determine one or more of cracking, creaking, and/or implant seating.

The system may include a display. The display may be configured to display an indication of one or more of the sound waves applied at the first end of the bone and/or the detected sound waves at the second end of the bone. For example, the display may display one or more sound metrics. The one or more sound metrics may be displayed with respect to a time and/or a position (e.g., location in a bone). The indication of the one or more sound waves may include a graph, for example of a sound metric over time and/or position. The user interface may include the display.

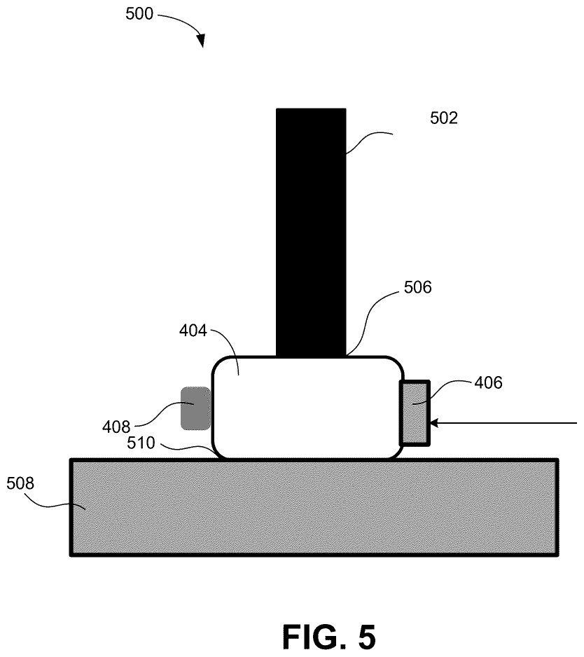

FIG. 5 is another sectional view of an example system 500 for applying and detecting sound in a bone. The system 500 may include a weight 502. The weight 502 may be configured to apply one or more of the force, stress, and/or load, for example to the bone 404. The weight 502 may apply a force to the bone 404 substantially perpendicular to a path of the sound waves (e.g., from the emitter 402 to the receiver 408). The weight may be disposed to contact the bone 404 at a third end 506. Alternatively, or additionally, an intervertebral implant may produce the force, stress, and/or load, for example to the bone 404 (e.g., instead of the weight 502).

A plate 508 may be configured to receive at least some of the force, for example applied by the weight 502. The plate 508 may be disposed to contact the bone 404 at a fourth end 510. The fourth end 510 may be opposite the third end 506 of the bone 504. The force applied by the weight 502 may be a desired (e.g., predetermined) force, for example selected by a user. The user may apply the plate 508 and/or the weight 502 to a bone to test the bone quality. Additionally, or alternatively the user may apply the plate 508 and/or the weight 502 to another material, for example a substitute for a bone, which may be used to determine a bone quality.

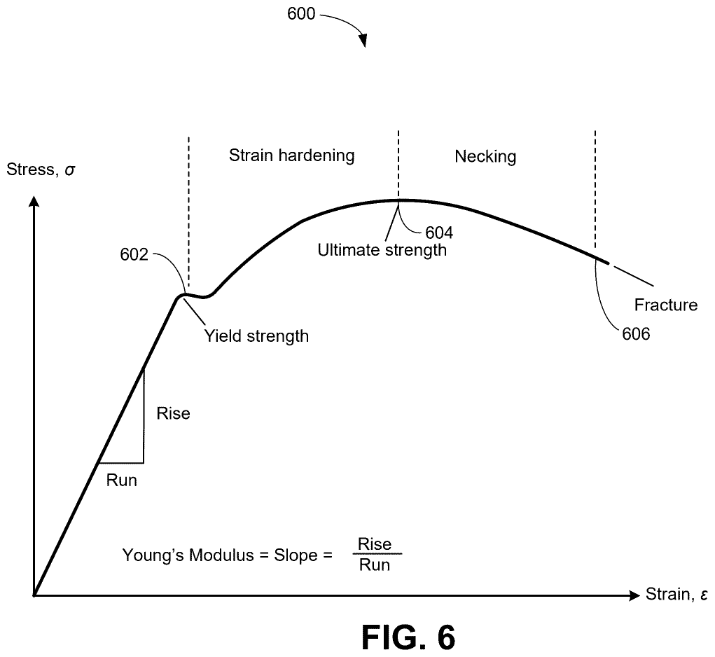

FIG. 6 is an example graph 600 of data indicating bone quality. The bone quality may include an indication of bone failure or fracture. The bone quality may be a function of the force applied, for example by the weight. The example graph 600 shows strain v. stress. As strain increases, stress likewise increases up to a yield strength point 602. As strain continues to increase beyond the yield strength point 602, stress increases up to an ultimate strength point 604. Strain hardening may occur, for example between the yield strength point 602 and the ultimate strength point 604. As strain continues to increase beyond the ultimate strength point 604, stress decreases. The increase in strain beyond the ultimate strength point 604 and/or the (e.g., corresponding) stress decrease may end at a fracture point 606. Necking may occur, for example between the ultimate strength point 604 and the fracture point 606.

A test to failure may be performed to determine data related to bone failure and/or fracture when under stress and/or strain. For example, the circuit may utilize a Cepstral technique to examine the sound data from the receiver (e.g., microphone). Additionally, or alternatively, the circuit may utilize wavelet detection to examine the sound information from the receiver. The circuit may utilize a technique to identify a non-linear region and/or to determine bone failure and/or fracture, for example before, during, and/or after a surgical procedure.

FIG. 7 is an example graph 700 of data generated using a bandpass filter. The user may apply the bandpass filter to the receiver (e.g., microphone) data, for example to filter unwanted frequencies. For example, the bandpass filter may filter out frequencies from other sources (e.g., not from the emitter). The example graph 700 shows time v. amplitude. At a first time 702, the emitter applies sound waves (e.g., the user may turn the emitter on). The amplitude at the first time 702 may have a large range. At a second time 704, the user may apply a first force (e.g., to the bone), for example the weight. The amplitude at the second time 704 may include a short increase in range, followed by a decrease in range, for example with respect to the amplitude at the first time 702.

At a third time 706, the user may apply a second force (e.g., weight) (e.g., to the bone). The second force may be greater than the first force. For example, the second force may be 40 pounds and/or the first force may be 20 pounds. The amplitude at the third time 706 may include a short increase in range, followed by a decrease in range, for example with respect to the amplitude at the first time 702 and/or the second time 704. The user and/or the circuit may apply different frequencies (e.g., at the same frequency), for example with different forces (e.g., weights).

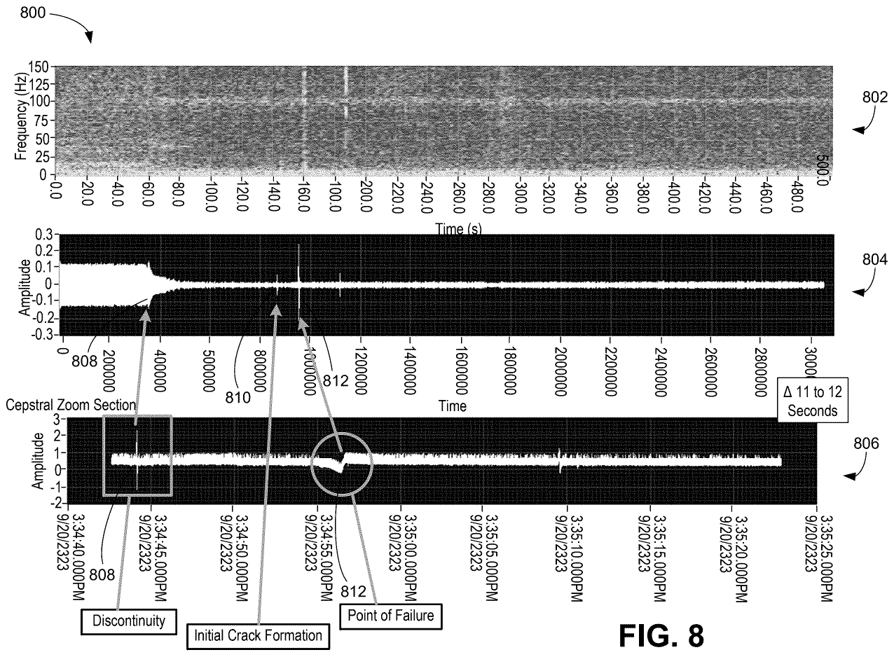

FIG. 8 includes example graphs 800 of data indicating spine failure. The example graphs may include data from a test procedure on a pig spine, for example at 800 Hz. A first graph 802 shows time v. frequency for (e.g., raw) output sound data (e.g., at the receiver). A second graph 804 shows time v. amplitude of filtered output sound data. A third graph 806 shows a section (e.g., Cepstral) of the second graph 804.

There may be a discontinuity point 808, where the amplitude includes a short increase in range, followed by a decrease in range. The third graph 806 shows the discontinuity point 808 on a smaller time scale. There may be an initial crack formation point 810, where the amplitude includes a short increase in range. There may be a point of failure 812, where the amplitude includes a short increase in range, followed by a decrease in range. The third graph 806 shows the point of failure 812 on a smaller time scale.

The increase in amplitude may correspond to the initial crack formation point 810. For example, the circuit may determine that a crack has formed (e.g., initially) at an increase in amplitude. The circuit may determine that the crack has formed based on a magnitude of the amplitude (e.g., the magnitude of the increase in amplitude). An increase in amplitude (e.g., a larger increase in amplitude) may correspond to the point of failure 812. The circuit may determine that the bone has failed based on a magnitude of the amplitude, for example a magnitude greater than the magnitude associated with the initial crack formation point 810. The circuit may determine a discontinuity, crack formation (e.g., initial), and/or point of failure based additionally, or alternatively, on a frequency magnitude and/or impedance magnitude. The circuit may determine a discontinuity, crack formation (e.g., initial), and/or point of failure based on a difference of amplitude, frequency, and/or impedance, for example between the emitted sound wave and the applied sound wave.

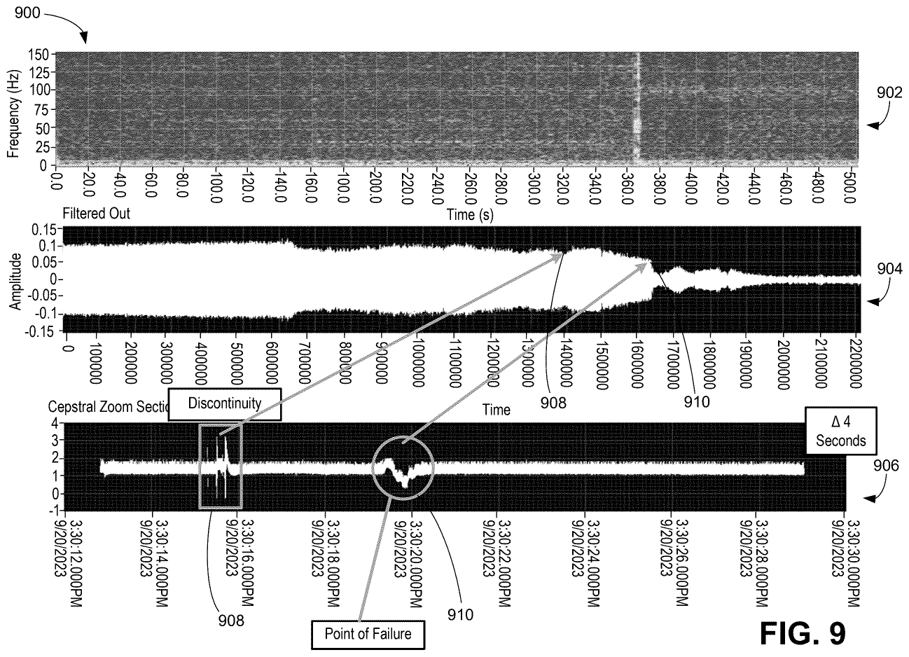

FIG. 9 includes additional example graphs 900 of data indicating spine failure. The example graphs may include data from a test procedure on a pig spine, for example at 800 Hz. A first graph 902 shows time v. frequency for (e.g., raw) output sound data (e.g., at the receiver). A second graph 904 shows time v. amplitude of filtered output sound data. A third graph 906 shows a section (e.g., Cepstral) of the second graph 904.

There may be a discontinuity point 908, where the amplitude includes a short increase in range, followed by a decrease in range. The third graph 906 shows the discontinuity point 908 on a smaller time scale. There may be a point of failure 910, where the amplitude includes a short increase in range, followed by a decrease in range. The third graph 906 shows the point of failure 912 on a smaller time scale. The user may utilize data and/or a graph (e.g., of data) to plan a (e.g., surgical) procedure.

What is claimed is:

1. A system comprising:
an emitter configured to generate and apply sound waves to a first end of a bone;
a receiver configured to detect the sound waves at a second end of the bone after the sound waves propagate through the bone; and
a circuit configured to compare the sound waves applied at the first end of the bone to the detected sound waves at the second end of the bone to determine an indication of bone quality.

2. The system of claim 1, wherein the emitter comprises at least one of a surface transducer, a sound emitter, a speaker, or a piezoelectric device.

3. The system of claim 1, wherein the receiver comprises at least one of a microphone, a piezoelectric device, or an accelerometer.

4. The system of claim 1, wherein the indication of bone quality comprises an indication of at least one of splintering, fracturing, cracking, or breaking, and wherein the circuit is further configured to determine the indication of bone quality based on the comparison of the sound waves applied at the first end of the bone to the detected sound waves at the second end of the bone.

5. The system of claim 1, wherein the circuit is further configured to determine the indication of bone quality based on an acoustic impedance of the sound waves propagated through the bone.

6. The system of claim 5, wherein the acoustic impedance of the sound waves is a function of a force, stress, or load applied to the bone.

7. The system of claim 1, further comprising an attachment mechanism configured to connect the receiver and the emitter to the bone.

8. The system of claim 7, wherein the attachment mechanism comprises at least one of a clamp, an adhesive, or a screw.

9. The system of claim 1, further comprising a display configured to display an indication of at least one of the sound waves applied at the first end of the bone or the detected sound waves at the second end of the bone.

10. The system of claim 1, wherein the circuit is further configured to determine the indication of bone quality based on at least one of the sound waves applied at the first end of the bone or the detected sound waves at the second end of the bone.

11. The system of claim 1, further comprising a filtering circuit comprising a 4.9 to 5.1 kHz bandpass filter.

12. A method of detecting sound in a bone comprising:
generating and applying sound waves to a first end of the bone;
detecting the sound waves at a second end of the bone after the sound waves propagate through the bone; and
comparing the sound waves applied at the first end of the bone to the detected sound waves at the second end of the bone to determine an indication of bone quality.

13. The method of claim 12, wherein generating and applying sound waves to the first end of a bone comprises generating the sound waves to propagate through the bone with at least one of a surface transducer, a sound emitter, a speaker, or a piezoelectric device.

14. The method of claim 12, wherein detecting the sound waves at the second end of the bone after the sound waves propagate through the bone comprises detecting the sound waves at the second end of the bone after the sound waves propagate through the bone with at least one of a microphone, a piezoelectric device, or an accelerometer.

15. The method of claim 12, wherein the indication of bone quality comprises an indication of at least one of splintering, fracturing, cracking, or breaking, and wherein the method further comprises determining the indication of bone quality based on the comparison of the sound waves applied at the first end of the bone to the detected sound waves at the second end of the bone.

16. The method of claim 12, further comprising determining the indication of bone quality based on an acoustic impedance of the sound waves propagated through the bone.

17. The method of claim 16, wherein the acoustic impedance of the sound waves is a function of a force, stress, or load applied to the bone.

18. The method of claim 12, further comprising attaching at least one of an emitter and a receiver to the bone with at least one of a clamp, an adhesive, or a screw.

19. The method of claim 12, further comprising displaying an indication of at least one of the sound waves applied at the first end of the bone or the detected sound waves at the second end of the bone.

20. The method of claim 12, further comprising determining the indication of bone quality based on at least one of the sound waves applied at the first end of the bone or the detected sound waves at the second end of the bone.
